(19) United States
(12) Patent Application Publication
(10) Pub. No.: US 2020/0411149 A1
(43) Pub. Date: Dec. 31, 2020

(54) FALL REPORTING

(71) Applicant: Hill-Rom Services, Inc., Batesville, IN (US)

(72) Inventors: Chiew Yuan Chung, Singapore (SG); Stacey A. Fitzgibbons, Dewitt, NY (US); Kristen Keaton Lightcap, Cary, NC (US); Matthew McCormick Riordan, Apex, NC (US); Yuan Shi, Singapore (SG); Eugene Urrutia, Durham, NC (US); Lori Ann Zapfe, Milroy, IN (US)

(21) Appl. No.: 16/911,486

(22) Filed: Jun. 25, 2020

Related U.S. Application Data
(60) Provisional application No. 62/868,386, filed on Jun. 28, 2019.

(51) Int. Cl. G16H 15/00; G16H 40/20; G16H 10/60; G16H 40/67; G16H 50/30; G16H 50/70; H04L 29/06; A61B 5/11; A61B 5/0205; A61B 5/00; H04L 29/08

(52) U.S. Cl. CPC G16H 15/00; G16H 40/20; G16H 10/60; G16H 40/67; G16H 50/30; G16H 50/70; A61B 5/021; A61B 5/1117; A61B 5/0205; A61B 5/6892; A61B 5/7278; A61B 5/0022; H04L 67/10; H04L 63/0815

(57) ABSTRACT

A fall reporting system gathers, extracts, and compiles data from multiple electronic sources to generate reports regarding patient falls. Individual patient investigation reports are automatically generated when a device detects a patient fall. Additionally, the system can aggregate data from multiple patients to provide facility investigation reports for a unit, department, or entire healthcare facility. Reports can be customized to filter and visualize falls-related data. The system can be cloud based or part of a local area network. The system accessible via a webportal that provides a single sign-on configuration application.

### RELATED APPLICATION(S)

This patent application claims the benefit of U.S. Patent Application Ser. No. 62/868,386 filed on Jun. 28, 2019, the entirety of which is hereby incorporated by reference.

### BACKGROUND

Patients in care facilities, such as hospitals, clinics, nursing homes or the like, are often in compromised medical conditions. Injuries sustained by patients due to falls in a care facilities result in significant healthcare costs. In an effort to prevent such injuries, various protocols are implemented to mitigate the risks. For example, patients who are at risk of falling when moving unassisted may be identified as fall risks, and certain protocols may be implemented to reduce the opportunity for the patients to move about the room unassisted.

Healthcare facilities implement various protocols for preventing falls and reporting falls when they occur. The extraction of information regarding compliance with these requirements is time-consuming, prone to human error, and not efficient. Additionally, information on the circumstances surrounding patient falls should be extracted promptly so that caregivers can better understand falls risk factors, monitor falls protocol compliance status, and minimize hospital costs for treatment of injuries that result from falls.

### SUMMARY

Embodiments of the disclosure are directed to a fall reporting system and methods of generating fall investigation reports.

In one aspect, a system for generating fall reports for a healthcare facility includes at least one processor and memory encoding instructions. When the instructions are executed by the at least one processor, it causes the at least one processor to: receive data from one or more electronic information systems and patient monitoring devices associated with the healthcare facility; extract falls-related data for at least one patient within the healthcare facility; automatically generate a patient investigation report based on the falls-related data for the at least one patient; and communicate the patient investigation report to at least one computer workstation.

In another aspect, one or more computer-readable media have computer-executable instructions embodied thereon that, when executed by one or more computing devices, cause the computing devices to: receive data from one or more electronic information systems and patient monitoring devices associated with a healthcare facility; receive a notification that a patient was involved in a fall within the healthcare facility; extract falls-related data for the patient from one or more of an electronic medical record system, a hospital information system, and a patient monitoring device; receive input of report parameters for a patient investigation report; automatically generate the patient investigation report based on the falls-related data for the patient and the report parameters; and communicate the patient investigation report to at least one computer workstation and an electronic medical record associated with the at least one patient.

In yet another aspect, computer-implemented method of generating fall reports for a healthcare facility comprises: receiving, at a computing device, data from one or more electronic information systems and patient monitoring devices associated with the healthcare facility; extracting falls-related data for at least one patient within the healthcare facility; automatically generating a patient investigation report based on the falls-related data for the at least one patient; communicating the patient investigation report to at least one computer workstation; aggregating, at the computing device, falls-related data for all patients within the healthcare facility, including the at least one patient; generating and displaying a user interface on the at least one computer workstation, the user interface comprising one or more parameter configuration boxes and a time configuration box; receiving selections of report parameters through the one or more parameter configuration boxes and the time configuration box on the user interface; filtering the aggregated falls-related data based on the selected report parameters; automatically generating a facility investigation report based on the filtered data; and displaying the facility investigation report on the at least one computer workstation, the facility investigation report comprising a chart area displaying one or more visualizations of the filtered data.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 is a block diagram schematically illustrating a healthcare facility that includes a fall reporting system.

FIG. 2 is a block diagram schematically illustrating the inputs and outputs and communications of the fall reporting system of FIG. 1.

FIG. 3 is a schematic block diagram of the fall reporting system of FIG. 1.

FIG. 4 is a schematic block diagram of an example computing device usable to implement aspects of the fall reporting system of FIG. 1.

FIG. 5 is a flow chart illustrating an example method of generating fall reports.

FIG. 6 illustrates an example facility fall investigation report.

FIG. 7 illustrates another example facility fall investigation report.

FIG. 8 illustrates another example facility fall investigation report.

FIG. 9 illustrates another example facility fall investigation report.

FIG. 10 illustrates another example facility fall investigation report.

FIG. 11 illustrates another example facility fall investigation report.

FIG. 12 illustrates another example facility fall investigation report.

FIG. 13 illustrates another example facility fall investigation report.

FIG. 14 illustrates another example facility fall investigation report.

FIG. 15 illustrates another example facility fall investigation report.

FIG. 16 illustrates an example patient fall investigation report.

FIG. 17 illustrates another example patient fall investigation report.

### DETAILED DESCRIPTION

The present disclosure is directed to systems and methods for automatically reporting fall events in a healthcare facility.

An example fall reporting system gathers, extracts, and compiles data from multiple electronic sources to generate reports regarding the context related to a patient fall. These reports can enable healthcare providers to better understand risk factors related to falls, monitor hospital falls protocol compliance status, and prevent future falls. Various rules define how information is populated into fields of a report and the collected data is visualized in one or more graphs. Reports can be generated for individual patients—particularly following a fall event. Additionally, the system can aggregate data from multiple patients to provide reports for a unit, department, or entire healthcare facility.

Data collected by the system is stored in a cloud computing environment. Reports can be accessed from a local web server and be displayed on a user interface. Users can customize the reports by making selections of report parameters through the user interface. In some embodiments, patient reports regarding fall risk can display risk-based alerts to caregivers and indicate when SBAR handoffs were sent and received.

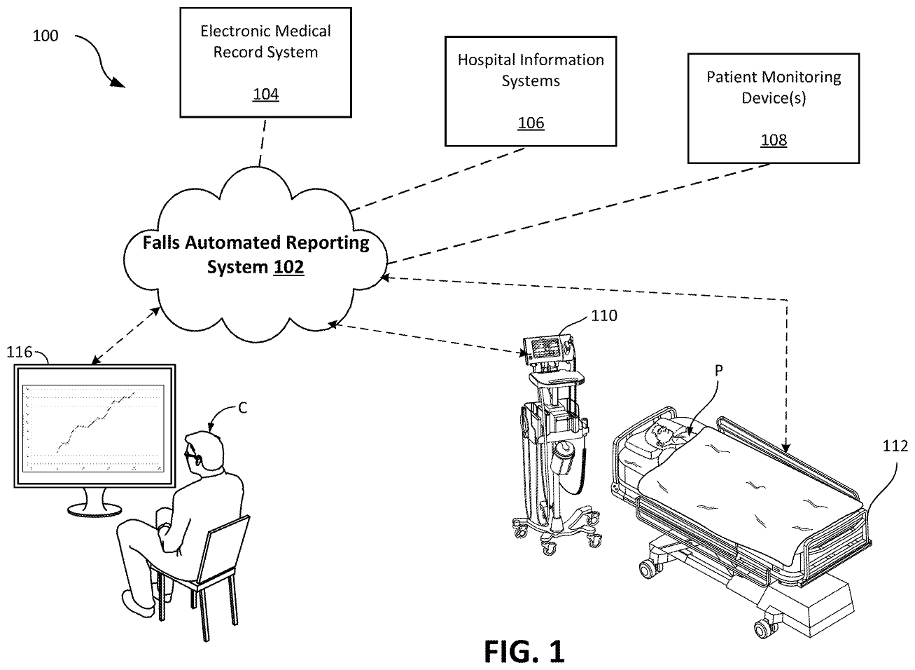

FIG. 1 is a schematic diagram illustrating a healthcare facility 100 that includes a fall reporting system 102. The fall reporting system 102 operates to gather information from multiple sources within the healthcare facility 100 to automatically generate reports about incidents of patients falling.

In the example of FIG. 1, the fall reporting system 102 communicates with an electronic medical record system 104 and hospital information systems 106. Information regarding particular patients P can be gathered from their electronic medical records and from other records stored in hospital information systems 106. Examples of such information include prescribed medications, recent and planned medical procedures, and family medical history. Other examples of information stored in electronic medical records can include dynamic data such as vitals sign readings and lab results.

The electronic medical record system 104 stores a plurality of electronic medical records (EMRs). Each EMR contains the medical and treatment history of a patient admitted to the healthcare facility 100. Examples of electronic medical records systems 104 include those developed and managed by Epic Systems Corporation, Cerner Corporation, Allscripts, and Medical Information Technology, Inc. (Meditech).

Examples of hospital information systems 106 include Admission, Discharge, and Transfer (ADT) systems, lab systems, medication systems, and other hospital related systems. An ADT system provides real-time information on each patient admitted to the healthcare facility 100 including the patient's name, address, gender, room assignment within the healthcare facility 100, the date and time when admitted to and discharged from the healthcare facility 100, and whether the patient has been transferred to another room or department within the healthcare facility 100. The lab system monitors patient samples and lab results. The medication system monitors the medications prescribed to each patient within the healthcare facility 100.

In the example of FIG. 1, the fall reporting system 102 is also in communication with one or more patient monitoring devices 108, which can include a vital signs monitoring device built into a bedside computer 110 and a patient support device such as a smart bed 112. These devices are associated with a particular patient P. One fall reporting system 102 can be communicating with multiple devices that are monitoring multiple patients. In some embodiments, one fall reporting system 102 monitors information from devices for all patients within a healthcare facility 100. Smart beds 112 can measure a patient's weight and record heart rate and respiratory rate of a patient P. Alarms or alerts can be communicated to caregivers C when it is detected that the patient P is exiting the bed without authorization. Examples of smart beds 112 include Centrella® Smart+ bed, Progressa® bed system, or VersaCare® Med Surg Bed, each available from Hill-Rom Services, Inc., Batesville, Ind. Load cells can be used to monitor ingress, egress, and patient movement on a bed. An example of a vital signs monitoring device 110 includes the Connex® Vital Signs Monitor available from Welch Allyn, Inc., Skaneateles Falls, N.Y.

Examples of other patient monitoring devices 108 include vitals monitors, mattress pad devices, and nurse call systems. Some patient monitoring devices 108 are configured to record patient vital signs such as blood oxygen level and heart rate. For example, the vitals monitor can be used to take vital signs such as temperature, heart rate, respiratory rate, blood pressure, pulse oximetry, and the like. In some examples, the vitals monitor is a monitor that can take readings both continuously and at intervals, such as the Connex® vitals sign monitor available from Welch Allyn Inc., Skaneateles Falls, N.Y. The mattress pad device is configured to be placed under the mattress of a bed in the healthcare facility 100, and continuously monitors heart rate, respiratory rate, and motion to help identify early detection of patient deterioration, prevent falls, and prevent pressure ulcers. In some examples, the mattress pad device is an EarlySense® system.

The fall reporting system 102 also communicates with one or more computer workstations 116. These workstations 116 are utilized by caregivers C within the healthcare facility 100 to monitor various aspects of the healthcare facility 100 including information regarding falls. Reports generated by the fall reporting system 102 can be viewed and manipulated on the computer workstation 116.

The fall reports are populated with data acquired by the fall reporting system 102 from the smart bed 104, bedside computer 110, patient monitoring device(s) 108, electronic medical records system 104, and hospital information systems 106. The fall reports allow caregivers to track hospital falls protocols compliance. Data visualization and actionable insights aid caregivers C in prioritizing falls interventions. The fall reports can be used to determine the pain points of the healthcare facility 100, and prioritize areas for improvement. In some embodiments, the fall reporting system 102 may be part of a larger reporting system or may be capable of providing reports on other types of events within the healthcare facility 100.

In one embodiment, the fall reporting system 102 is a cloud-based system that is hosted over the Internet. In this example embodiment, the fall reporting system 102 is accessible from the workstation 116 via a web portal that provides a single sign-on configuration application.

In an alternative embodiment, the fall reporting system 102 is part of a local area network and is stored onsite in the healthcare facility 100. In this example embodiment, the fall reporting system 102 is accessible from the workstation 116 via an intranet portal that provides a single sign-on configuration application.

In one example embodiment, the workstation 116 is a stationary desktop computer. In alternative example embodiments, the workstation 116 is a portable computing device such as a smartphone, tablet computer, and the like. Although only one workstation 116 is depicted in FIG. 1, it is contemplated that the healthcare facility 100 can include a plurality of workstations 116 that are accessible by a plurality a caregivers C.

A caregiver can customize the fall reports generated by the fall reporting system 102 by selecting one or more options from one or more menus including at least a parameter configuration box and a time configuration box. The caregiver can customize the fall reports by selecting which type(s) of data to display, for which period of time, for which units/departments within the healthcare facility 100, and for which types of patients.

The fall reporting system 102 also provides back office settings where an administrator can configure the rules for determining high/medium/low patient risk, and the types of alerts that are sent based on the determined risk. The back office settings can also allow the administrator to configure the rules for granting access to the fall reports.

The fall reporting system 102 communicates with the electronic medical record system 104, hospital information systems 106, patient monitoring and support devices 108, bedside computer 110, smart bed, and workstation 116 through a wireless connection, a wired connection, or a combination of wireless and wired connections. Examples of wireless connections include Wi-Fi communication devices that utilize wireless routers or wireless access points, cellular communication devices that utilize one or more cellular base stations, Bluetooth, ANT, ZigBee, medical body area networks, personal communications service (PCS), wireless medical telemetry service (WMTS), and other wireless communication devices and services.

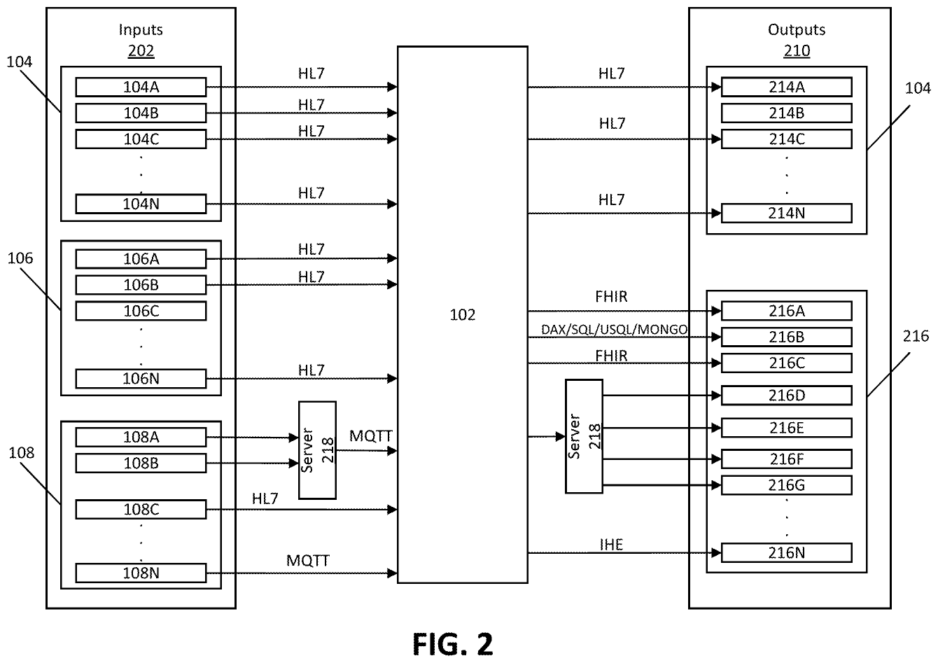

FIG. 2 is a block diagram schematically illustrating the inputs 202 and outputs 210 of the fall reporting system 102. As shown in FIG. 2, the fall reporting system 200 retrieves inputs 104a-104n from the electronic medical record system 104, inputs 106a-106n from the hospital information systems 106, and inputs 108a-108n from the patient monitoring devices 108.

The inputs 104a-104n are directly retrieved using a Health Level Seven International (HL7) messaging protocol that allows the information to be shared and processed in a uniform and consistent manner. Similarly, the inputs 106a-106n are directly retrieved over the HL7 data protocol. In some examples, the inputs 108a-108n are directly retrieved using HL7 data standards. For example, inputs from the vitals monitor can be retrieved using the HL7 standard. In other examples, the inputs 108a-108n are retrieved using a Message Queuing Telemetry Transport (MQTT) messaging protocol. For example, inputs from the mattress pad devices such as the EarlySense® system can be communicated over the MQTT standard. In some further examples, the inputs 108a-108n are indirectly retrieved using a secondary server 218. For example, the fall reporting system 102 can communicate with a secondary server 218 such as the SmartSync™ system from Hill-Rom Services, Inc. to retrieve data from the beds such as the Centrella® Smart+ bed, Progressa® bed system, or VersaCare® Med Surg Bed.

The fall reporting system 102 optionally generates outputs 214a-214n for the electronic medical record system 104 and outputs 216a-216n for clinical user interfaces 216. At least one of the outputs 216a-216n is a fall report on a web portal or intranet portal accessible via the workstation 116.

Outputs 214a-214n are directly sent to the electronic medical record system 104 using the HL7 messaging protocol. Outputs 216a-216n are directly sent to a clinical user interface 216 using Fast Healthcare Interoperability Resources (FHIR), Integrating the Healthcare Enterprise (IRE), or DAX/SQL/USQL/MONGO data formats. In some examples, the outputs 216a-216n are indirectly sent to a clinical user interface 216 using a secondary server 218.

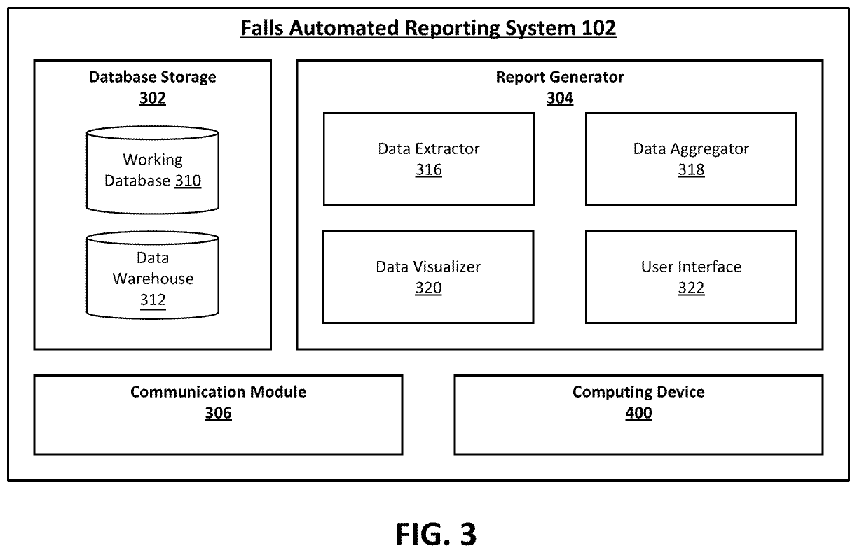

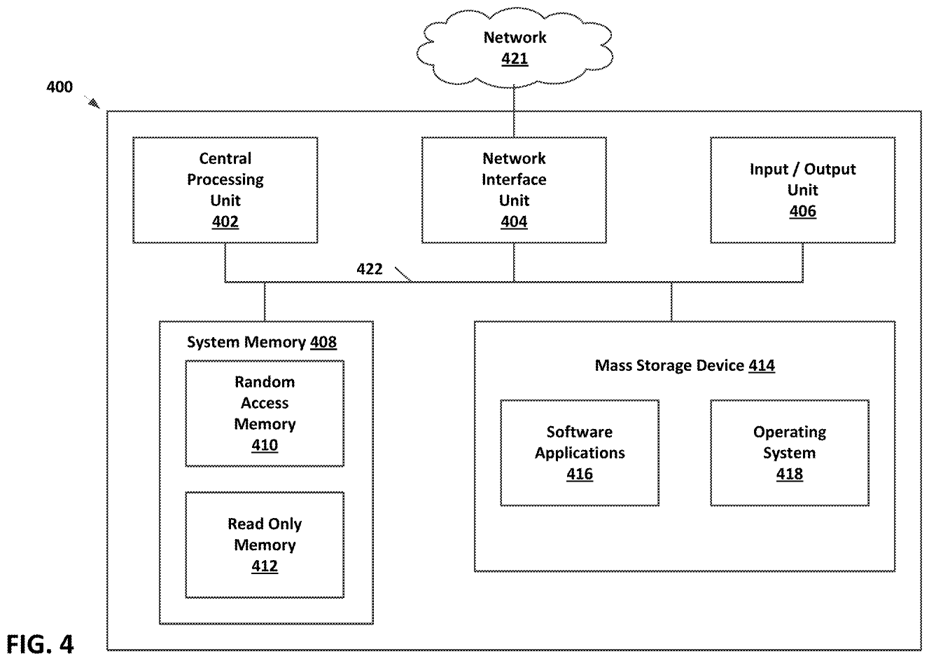

FIG. 3 is a block diagram illustrating an embodiment of the fall reporting system 102. As shown in FIG. 3, the fall reporting system 102 includes database storage 302, a report generator 304, a communication module 306, and a computing device 400. The database storage 302 stores the data retrieved from the electronic medical record system 104, hospital information systems 106, and patient monitoring devices 108. The report generator 304 uses the data stored in the database storage 302 to generate the fall reports. The communication module 306 enables the fall reporting system 102 to communicate with the electronic medical record system 104, hospital information systems 106, patient monitoring devices 108, and workstation 116 in the healthcare facility 100. The computing device 400 is described in more detail with reference to FIG. 4.

The database storage 302 includes a working database 310 and a separate data warehouse 312. The working database 310 temporarily stores the data from the electronic medical record system 104, hospital information systems 106, and patient monitoring devices 108. The data in the working database 310 is used to trigger one or more rules and/or alerts. Protocols for the healthcare facility can be stored in the working database 310 to enable the fall reporting system 102 to determine when alerts should be triggered. For example, patient risk scores and early warning scores (EWS) when above a predetermined threshold trigger alerts for the caregiver to perform critical tasks. In certain examples, the working database 310 is a clinical data repository (CDR). The data in the working database 310 is removed or erased after a predetermined event or period of time. For example, the data in the working database 310 is removed upon the patient's discharge from the healthcare facility 100 or upon a predetermined amount of time after the patient's discharge from the healthcare facility 100.

The data warehouse 312 stores the data long term from the electronic medical record system 104, hospital information systems 106, and patient monitoring devices 108. The patient data stored in the data warehouse 312 may be anonymized (de-identified) such that the data is not associated with any particular patient name or patient ID number. Data stored in the data warehouse 312 is used by the report generator 304 to populate the various fall reports disclosed herein.

The report generator 304 automatically generates patient investigation reports and can generate other facility investigation reports upon request. In the example of FIG. 3, the report generator 304 includes a data extractor 316, a data aggregator 318, a data visualizer 320, and a user interface 322.

The data extractor 316 operates to extract falls-related data from electronic medical records systems 104, hospital information systems 106, and patient monitoring devices 108. In some embodiments, data is extracted for individual patients to generate patient investigation reports. The extracted falls-related patient data can be stored in the working database 310 until it can be utilized to generate patient-specific reports. The falls-related data can also be anonymized and stored in the data warehouse 312 for use in generating facility investigation reports.

The data aggregator 318 operates to aggregate falls-related data for multiple patients for generating facility investigation reports. The falls-related data can be drawn from the data warehouse 312 so that the information is not specific to any particular patient and does not include any patient identifying information. Data can be aggregated for a unit, a department, or an entire healthcare facility.

The data visualizer 320 operates generate graphs and charts to visualize selected data relating to falls. Report parameters define the data that will be used to generate the graphs and charts. Examples of chart types include histograms, line graphs, bubble charts, and scatter plots. Visualizations can also include timelines and tables.

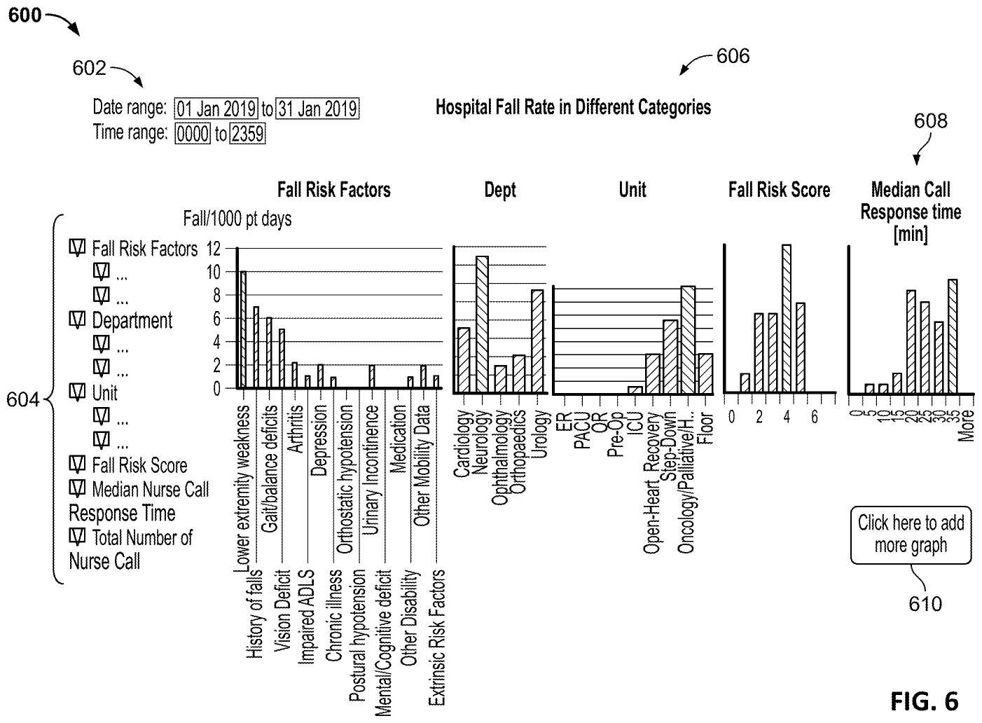

The user interface 322 operates to present a visual means for interaction with a user on a computer workstation. In some embodiments, the user interface 322 displays a time configuration box 602 and parameter configuration boxes 604 (discussed in FIG. 6) and receives input of report parameters. These report parameters define which falls-related data is going to be displayed in a visualization.

FIG. 4 is a block diagram illustrating an example of the physical components of a computing device 400. The computing device 400 could be any computing device utilized in conjunction with the fall reporting system 102 such as the computer workstation 116 or the bedside computer 110 of FIG. 1.

In the example shown in FIG. 4, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing device 400 further includes a mass storage device 414. The mass storage device 414 is able to store software instructions and data such as falls-related data extracted from electronic healthcare records.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media.

Computer-readable storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to various embodiments, the computing device 400 can operate in a networked environment using logical connections to remote network devices through a network 421, such as a wireless network, the Internet, or another type of network. The computing device 400 may connect to the network 421 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions and data. The software instructions include an operating system 418 suitable for controlling the operation of the computing device 400. The mass storage device 414 and/or the RAM 410 also store software instructions, that when executed by the CPU 402, cause the computing device 400 to provide the functionality discussed in this document. For example, the mass storage device 414 and/or the RAM 410 can store software instructions that, when executed by the CPU 402, cause the computing device 400 to generate fall reports.

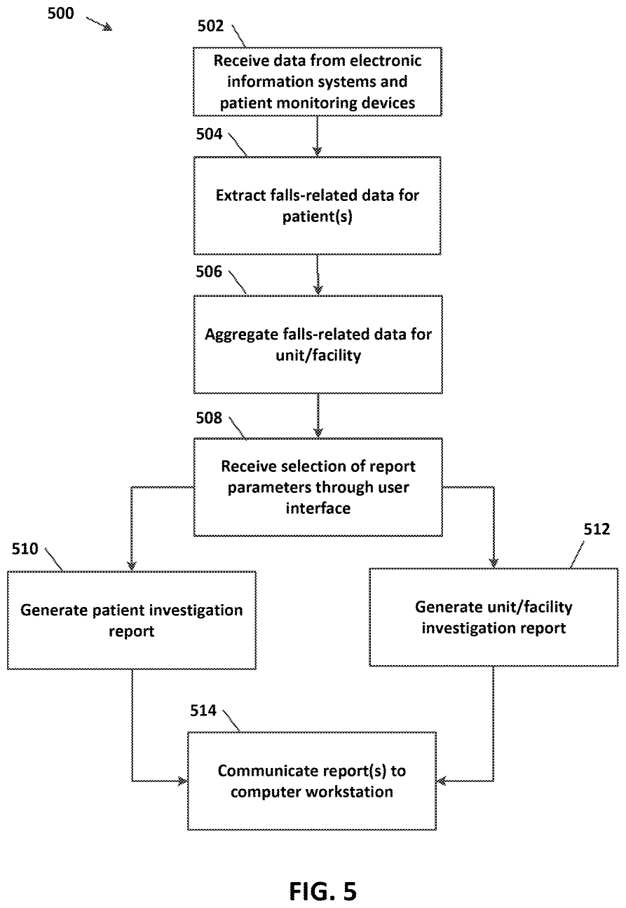

Referring now to FIG. 5, an example method 500 of automatically generating fall reports is described. The example method 500 can be performed, for example, using the fall reporting system 102 of FIGS. 1 and 2 described above.

At operation 502, data is received from electronic information systems and patient monitoring devices. The electronic information systems can include EMR systems, ADT systems, lab systems, medication systems, real-time location systems, and other hospital related systems. The patient monitoring devices can include vitals monitors, smart beds, and mattress pad devices.

At operation 504, falls-related data for one or more patients is extracted. If a particular time range is not specified, the time range for the data extraction defaults to the time between patient admission and discharge.

At operation 506, falls-related data for a unit or facility is aggregated from the patient-specific falls-related data that was extracted in operation 504. In some embodiments, the falls-related data is anonymized before it is aggregated.

At operation 508, report parameters are received through a user interface. The report parameters specify a time range and types of data that are to be displayed in a falls investigation report. In some embodiments, a user may select a time range specifically to include a time period in which a fall occurred. The report parameters also specify whether to generate a patient investigation report or a unit/facility investigation report. For patient investigation reports, the method proceeds to operation 510. For unit/facility investigation reports, the method proceeds to operation 512.

At operation 510, a patient investigation report is automatically generated. The patient investigation report includes visualizations of the data selected with the report parameters. The visualization can be modified by changing report parameters on the user interface. In some embodiments, the report is requested to include information about a particular patient fall. The report will reflect the circumstances in which the fall occurred as well as the events leading up to the fall. In some embodiments, the patient investigation report can include information about the events that occurred after the fall. The patient investigation report can include information indicating whether falls-related protocols were complied with for the patient. Patient investigation reports can also be configured to display risk-based alerts to caregivers. Patient investigation reports can further include information about when SBAR (Situation, Background, Assessment, Recommendation) handoffs occurred.

At operation 512, a unit or facility investigation report is generated. The data selected with the report parameters is visualized in one or more graphs or charts. The visualization can be modified by changing report parameters on the user interface.

At operation 514, the fall investigation report is communicated to at least one computer workstation. The patient investigation report can be viewed on the computer workstation by a caregiver to analyze what might have contributed to the fall and how a fall could be avoided in the future. The unit/facility investigation report can be viewed on the computer workstation by a caregiver or administrator to evaluate overall fall risk mitigation performance and make adjustments to protocols.

FIGS. 6-15 illustrate examples of facility fall investigation reports 600. For each view of the facility fall investigation reports 600, a date and/or time range is selected in a time configuration box 602. Additionally, other parameters are selected in the parameter configuration boxes 604. These other parameters can be used to narrow down the data shown based on criteria such as fall risk factors, department, and fall risk score. The time configuration box 602 and parameter configuration boxes 604 can be used to customize the reports via the user interface 322. A title 606 describes the content of the report 600. A chart area 608 provides a visualization of the data selected using the time configuration box 602 and parameter configuration boxes 604. The chart area 608 can include a combination of multiple graphs, as shown in the example of FIG. 6. Additional graphs can be added to the chart area 608 when input is received at the add graph button 610. In some examples, the facility fall investigation report 600 displays timeframe display configuration boxes 612 to customize how data is shown in the chart area 608. Additionally, a chart legend 614 can be displayed to clarify how the data is visualized in the chart area 608.

In the example of FIG. 6, the facility fall investigation report 600 displays a series of graphs in the chart area 608 that indicate how many falls occurred, broken down based on fall risk factors, department, unit, fall risk score, and median call response time.

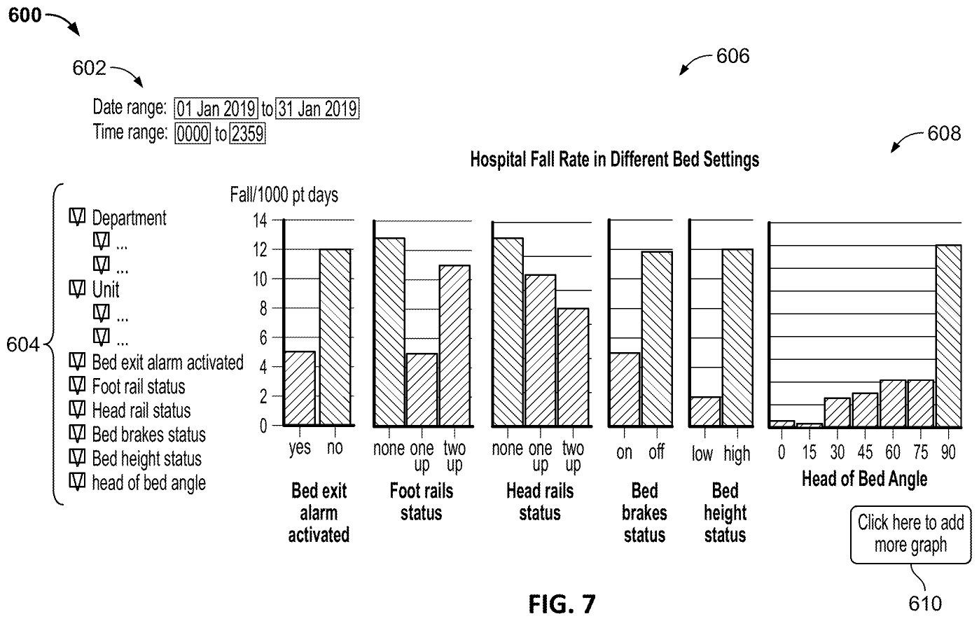

FIG. 7 illustrates an example of a facility fall investigation report 600 that shows hospital fall rates in different bed settings, as indicated by the title 606. Selections of the parameter configuration boxes 604 cause data for bed exit alarm activation, foot rail status, head rail status, bed brakes status, bed height status, and head of bed angle to be displayed in the chart area 608.

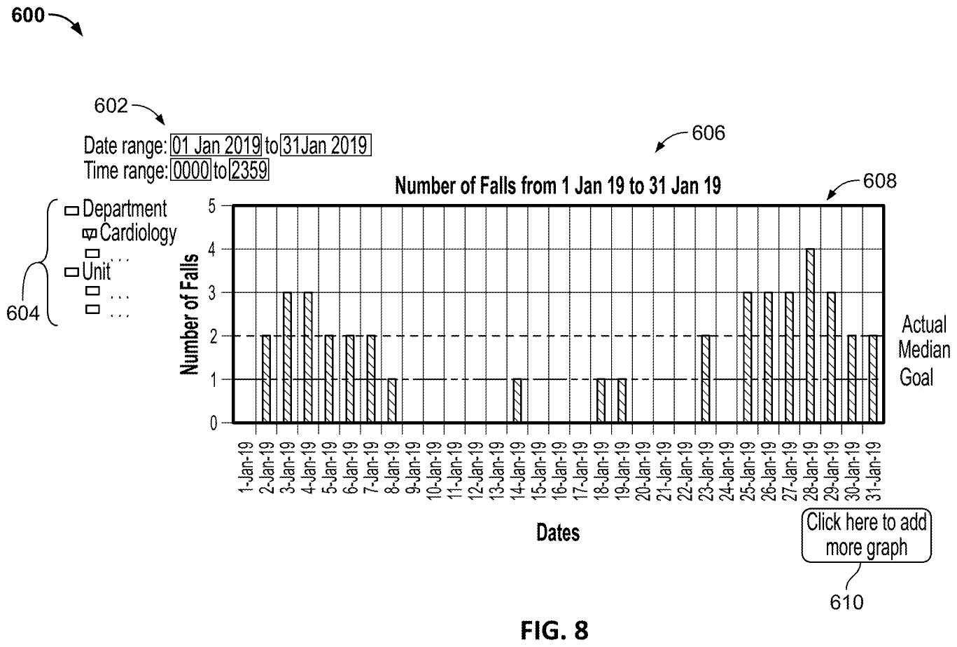

In the example facility fall investigation report 600 of FIG. 8, the chart area 608 shows data for the number of falls that occurred in cardiology at a given facility on each day within the time range selected in the time configuration box 602. The title 606 reflects the data selections for “Number of Falls from 1 Jan 19 to 31 Jan 19.” In this example, a dotted line for the goal number of falls for each day is shown as well as a dotted line reflecting the actual median number of falls.

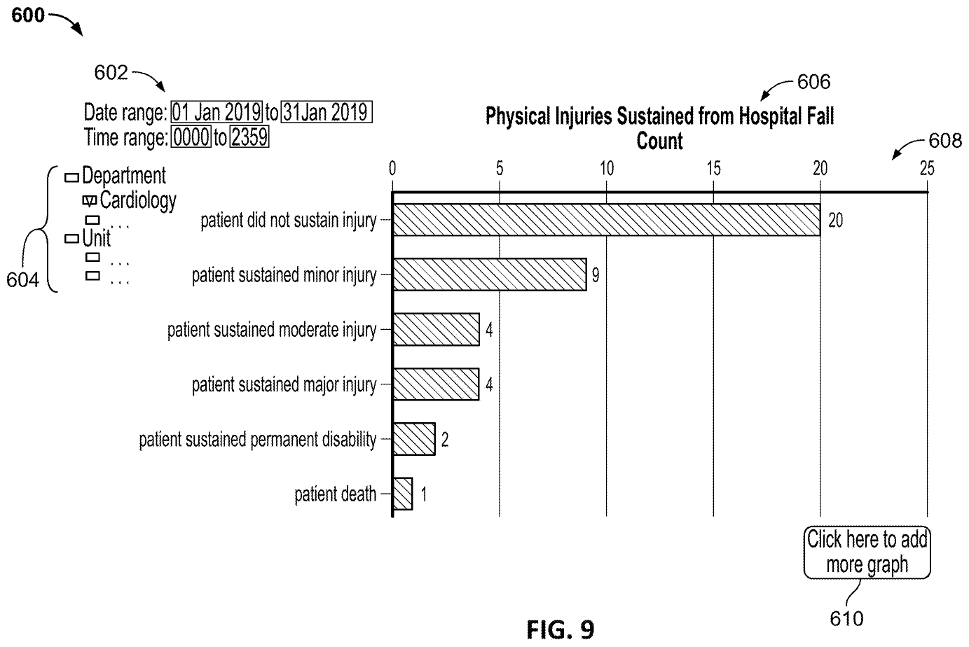

FIG. 9 illustrates an example facility fall investigation report 600 for “Physical Injuries Sustained from Hospital Fall” in the cardiology department. The data selected by the time configuration box 602 and parameter configuration box 604 is shown in a bar chart in the chart area 608.

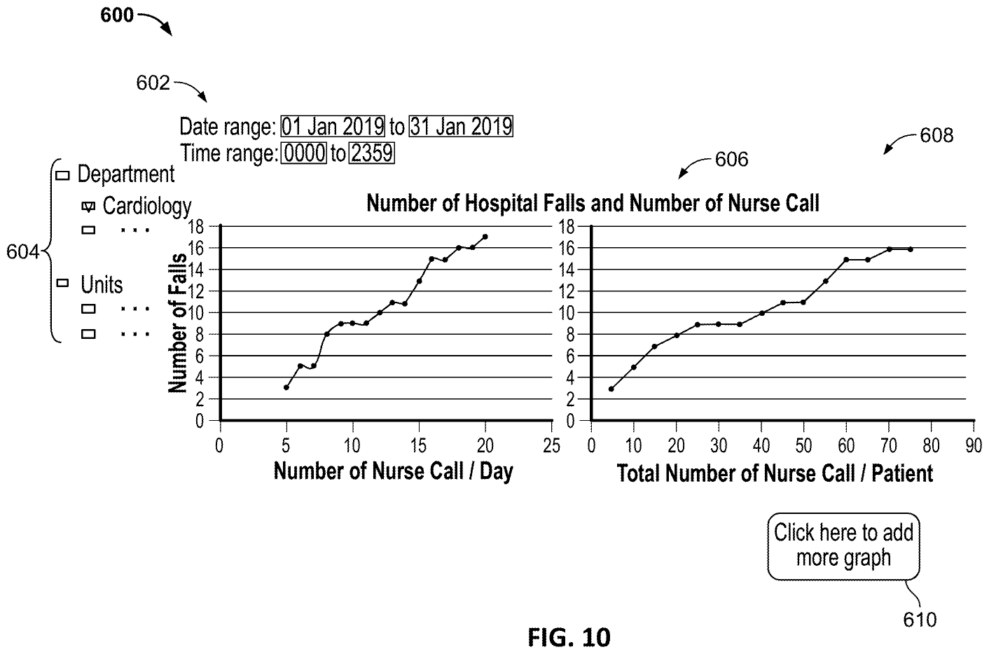

FIG. 10 illustrates an example facility fall investigation report 600 visualizing data for the cardiology department in January 2019, as indicated by the time configuration box 602 and parameter configuration box 604. Two line graphs in the chart area 608 show the number of hospital falls compared to the number of nurse calls. The graph on the left reflects the number of nurse calls per day and the graph on the right reflects the number of nurse calls per patient.

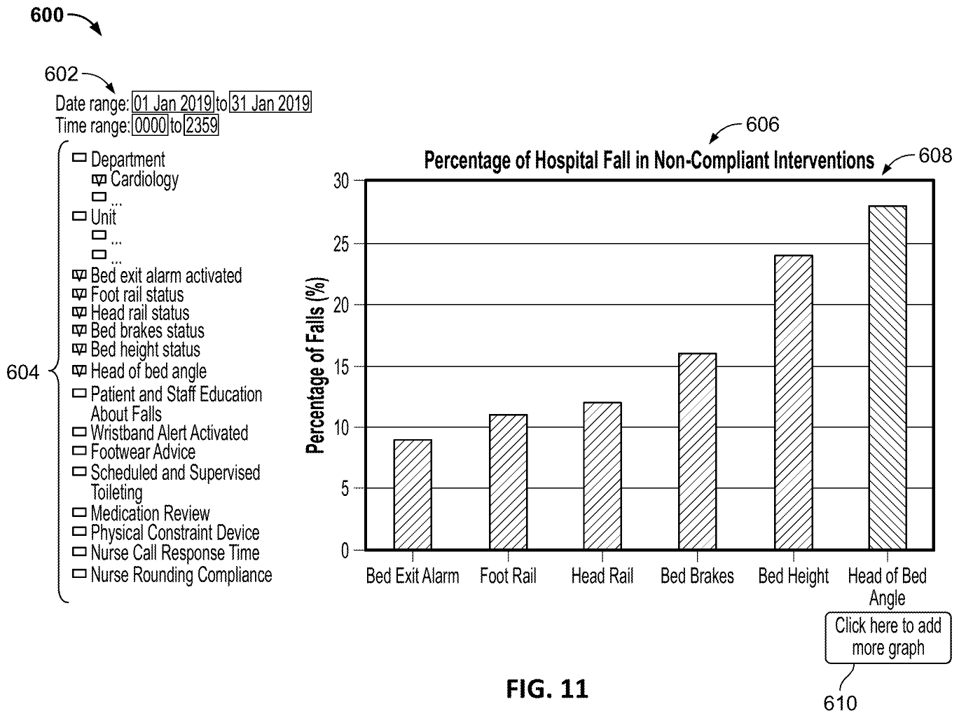

The facility fall investigation report 600 shown in FIG. 11 shows that the date range of Jan. 1, 2019 to Jan. 31, 2019 has been selected in the time configuration box 602. Selections in the parameter configuration box 604 have been made to display data for the cardiology department that relate to falls statistics for when the bed exit alarm is activated, whether the foot rails were in a compliant position, whether the head rails were in a compliant position, whether the bed brakes were engaged, the height status of the bed, and the angle of the head of the bed. The title 606 of this report is “Percentage of Hospital Fall in Non-Compliant Interventions.” The chart area 608 shows a histogram reflecting the percentage of falls that occurred in January 2019 and coincide with non-compliant interventions. This particular graph indicates that a non-compliant head angle of the bed coincided with the most falls.

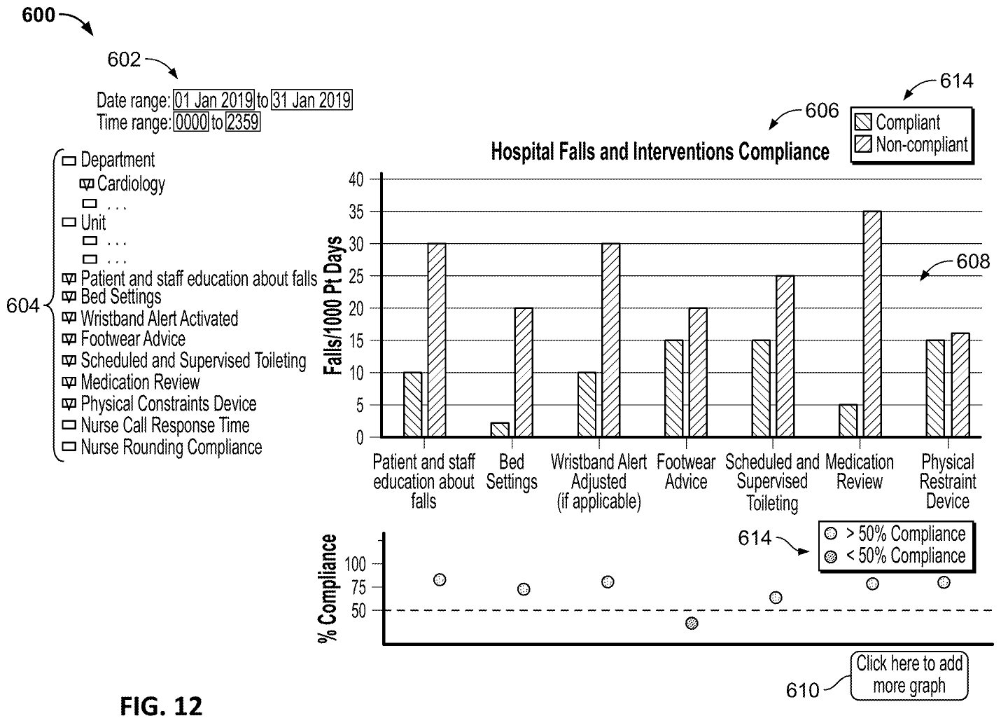

FIG. 12 illustrates an example facility fall investigation report 600 showing a comparison of the number of falls that occurred when interventions were compliant compared to non-compliant. Each graph within the chart area 608 has its own chart legend 614. Generally, the chart area 608 shows a histogram that indicates that more falls occur when interventions are not in compliance compared to when interventions are in compliance. The % compliance graph provides another visualization to further illustrate the trend.

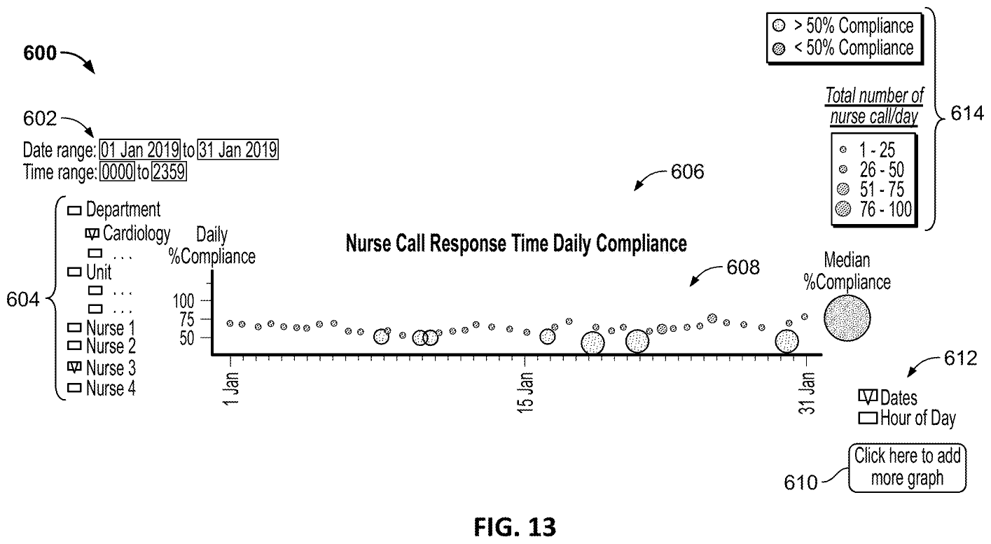

FIG. 13 illustrates another example of a facility fall investigation report 600. This report 600 focuses on a particular nurse within the cardiology department, as indicated in the parameter configuration box 604. The title 606 indicates that the data visualized in the chart area 608 relates to the daily % of compliance of that particular nurse to the response time protocols. The chart legend 614 indicates that larger circles represent more calls per day. The timeframe display configuration boxes 612 have been checked to show the data by date. The data visualized in the chart area 608 reflects a trend that when more nurse calls are made in a day, Nurse 3 is less compliant with the response time protocols.

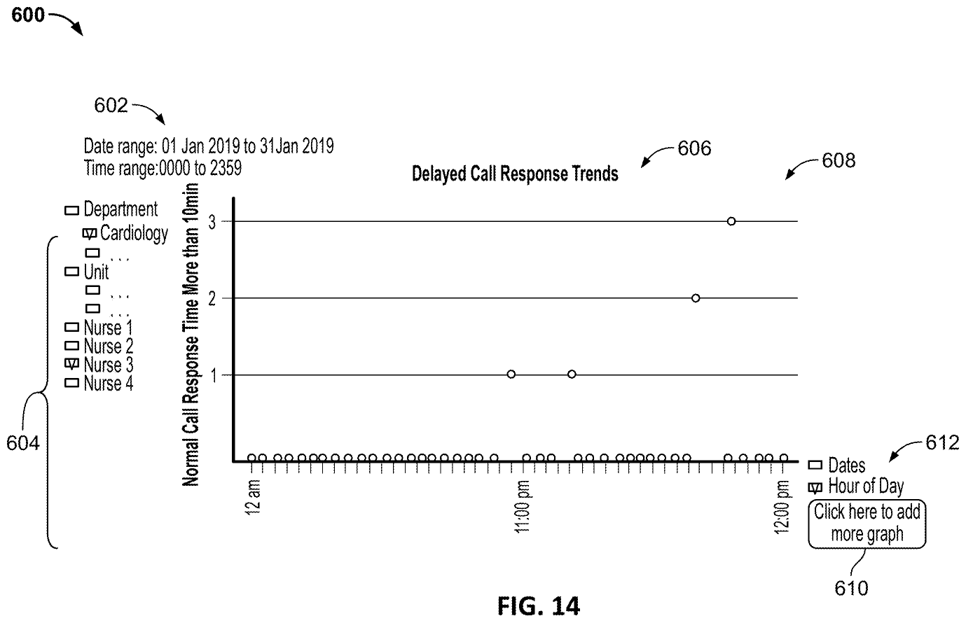

FIG. 14 illustrates another facility fall investigation report 600 that focuses on Nurse 3 in cardiology. As indicated by the title 606 and the selection made in the timeframe display configuration boxes 612, this visualization is looking at the call response time trends for Nurse 3 based on time of day.

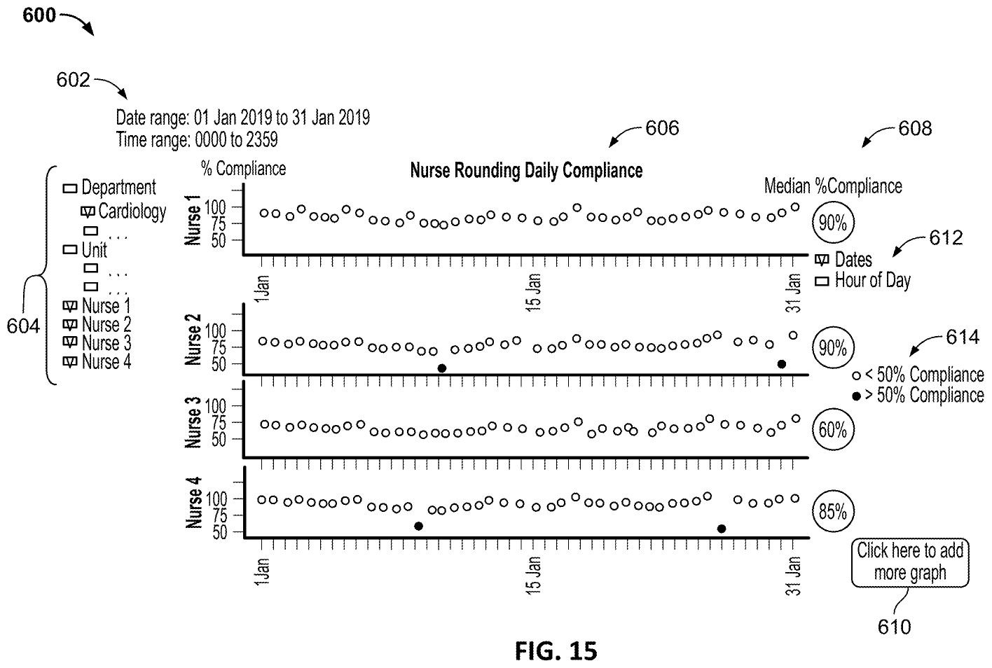

The facility fall investigation report 600 of FIG. 15 shows a visualization with the title 606 “Nurse Rounding Daily Compliance.” The parameter configuration box 604 indicates that the data shown is for four nurses in the cardiology department and the time configuration box 602 indicates that the data shown is for the month of January 2019. The chart area 608 shows four graphs—one for each nurse. As indicated by the timeframe display configuration boxes 612, the percentage of compliance with nurse rounding protocols is shown by day. The chart legend 614 indicates that for days when the nurse is over 50% compliant, a blue dot is displayed and when the nurse is under 50% compliant, a red dot is displayed. The chart area 608 also shows a median compliance figure for each nurse for that selected time period.

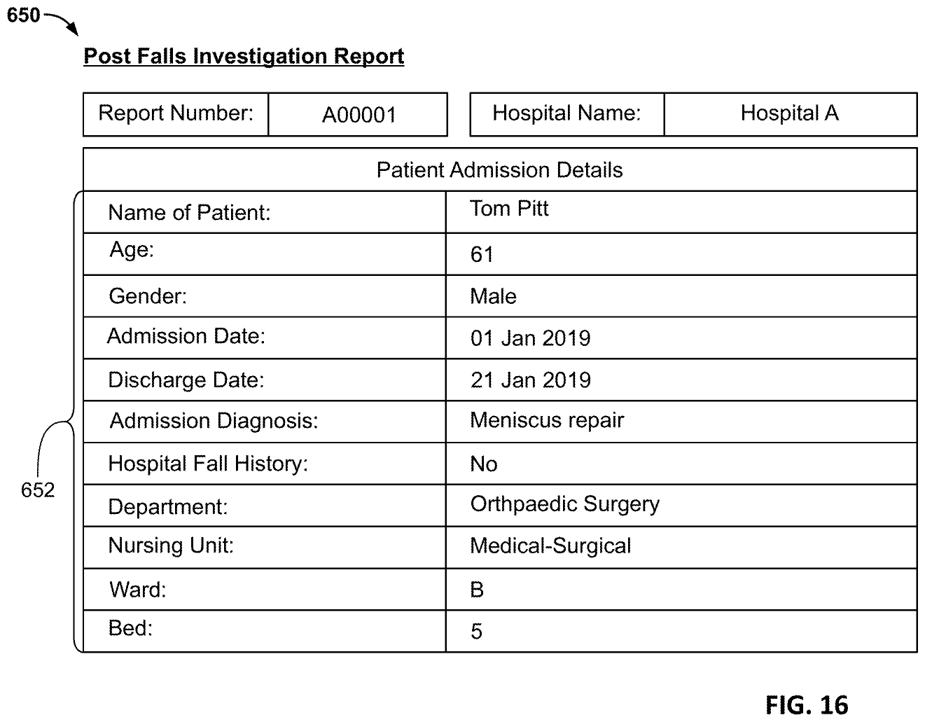

FIG. 16 illustrates an example patient fall investigation report 650. In this view, a report number and hospital name are listed at the top of the report. Patient details 652 are listed including name of patient, age, gender, admission date, discharge date, admission diagnosis, hospital fall history, department, nursing unit, ward identifier, and bed number.

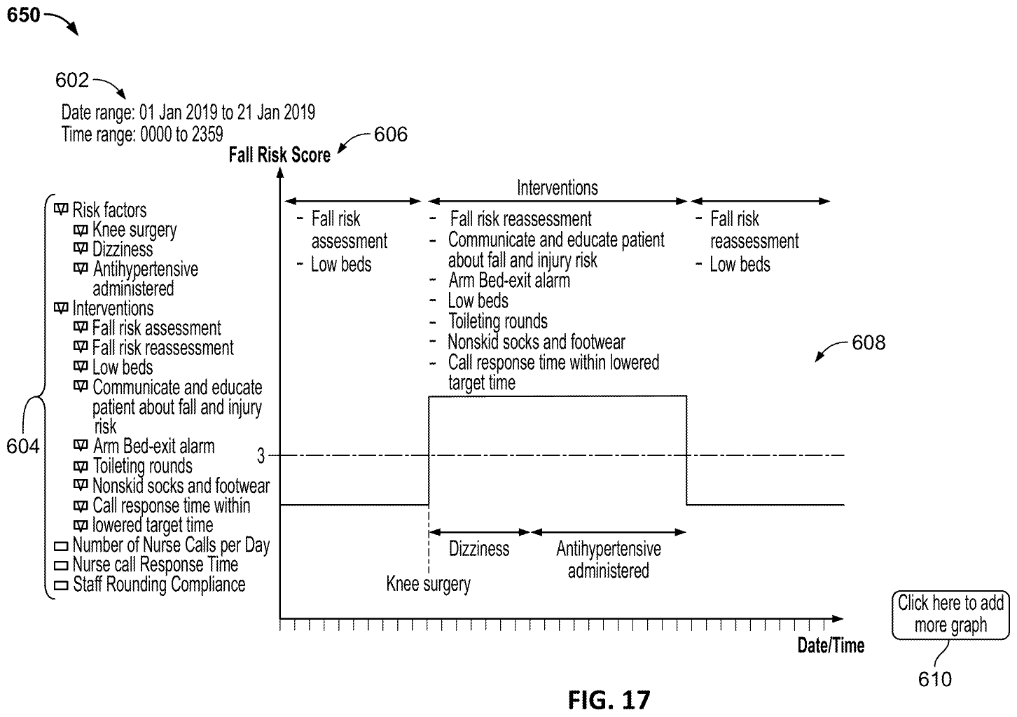

FIG. 17 shows another view of a patient fall investigation report 650. The time configuration box 602 indicates that the data shown is for Jan. 1, 2019 to Jan. 21, 2019. A time range reflecting the date of admission and date of discharge for the patient will be selected by default when report parameters are not specified by a user. The parameter configuration box 604 indicates risk factors and interventions will be displayed. These parameters can be customized through interactions with a user interface 322. The chart area 608 shows a graph illustrating how the patient's fall risk score is expected to change over 21 days. Interventions are listed at the top of the graph, where bold red interventions indicate non-compliance. The graph shows how the patient's risk factors contribute to a higher fall risk score. The dotted line indicates the threshold between low and high risk. In this example, the patient's highest risk of fall occurs in the days following knee surgery due to dizziness and administration of antihypertensive. Additional graphs could be added to the chart area 608 using the add graph button 610.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A system for generating fall reports for a healthcare facility, the system comprising:
at least one processor; and
memory encoding instructions which, when executed by the at least one processor, cause the at least one processor to:
receive data from one or more electronic information systems and patient monitoring devices associated with the healthcare facility;
extract falls-related data for at least one patient within the healthcare facility;
automatically generate a patient investigation report based on the falls-related data for the at least one patient; and
communicate the patient investigation report to at least one computer workstation.

2. The system of claim 1, wherein the memory encodes further instructions which, when executed by the at least one processor, cause the at least one processor to:
aggregate falls-related data for all patients within the healthcare facility;
generate and display a user interface on the at least one computer workstation, the user interface comprising one or more parameter configuration boxes and a time configuration box;
receive selections of report parameters through the one or more parameter configuration boxes and the time configuration box on the user interface;
filter the aggregated falls-related data based on the selected report parameters;
automatically generate a facility investigation report based on the filtered data; and
display the facility investigation report on the at least one computer workstation, the facility investigation report comprising a chart area displaying one or more visualizations of the filtered data.

3. The system of claim 1, wherein the system is a cloud based system that is accessible via a web portal that provides a single sign-on configuration application.

4. The system of claim 1, wherein the system is part of a local area network that is accessible via an intranet portal that provides a single sign-on configuration application.

5. The system of claim 1, wherein the electronic information systems comprise one or more of an electronic medical record (EMR) system, a nurse call system, an admit-discharge-transfer (ADT) system, a real-time location system, and a hospital information system.

6. The system of claim 1, wherein the patient monitoring devices comprise one or more of a vital sign monitor, a smart bed, a bedside computer, a mattress pad device, a blood pressure monitoring device, a blood oxygen monitoring device, and a heart rate monitoring device.

7. The system of claim 1, wherein the patient investigation report provides visualizations of patient data comprising one or more of fall risk score, bed exit alarm status, foot rail position, head rail position, bed brake status, bed height status, head of bed angle, load cell readings, and patient vital sign readings.

8. The system of claim 1, wherein the patient investigation report is generated using predefined, default parameter settings.

9. The system of claim 1, wherein the memory encodes further instructions which, when executed by the at least one processor, cause the at least one processor to:
generate and display a user interface on the at least one computer workstation, the user interface comprising one or more parameter configuration boxes and a time configuration box; and
receive input of one or more report parameters.

10. The system of claim 1, wherein the at least one computer workstation comprises one or more of a desktop computer, a tablet computer, a smart TV, and a smartphone.

11. The system of claim 1, wherein the data received from the one or more electronic information systems and patient monitoring devices associated with the healthcare facility is retrieved using a Health Level Seven International messaging protocol.

12. The system of claim 1, further comprising a database storage that includes anonymized data for populating the facility investigation reports.

13. The system of claim 1, wherein the patient investigation report is automatically generated when data is received indicating that the at least one patient was involved in a fall.

14. One or more computer-readable media having computer-executable instructions embodied thereon that, when executed by one or more computing devices, cause the computing devices to:
receive data from one or more electronic information systems and patient monitoring devices associated with a healthcare facility;
receive a notification that a patient was involved in a fall within the healthcare facility;
extract falls-related data for the patient from one or more of an electronic medical record system, a hospital information system, and a patient monitoring device;
receive input of report parameters for a patient investigation report;
automatically generate the patient investigation report based on the falls-related data for the patient and the report parameters; and
communicate the patient investigation report to at least one computer workstation and an electronic medical record associated with the at least one patient.

15. The computer-readable media of claim 14, wherein the electronic information systems comprise one or more of an electronic medical record (EMR) system, a nurse call system, an admit-discharge-transfer (ADT) system, a real-time location system, and a hospital information system.

16. The computer-readable media of claim 14, wherein the patient monitoring devices comprise one or more of a vital sign monitor, a smart bed, a bedside computer, a mattress pad device, a blood pressure monitoring device, a blood oxygen monitoring device, and a heart rate monitoring device.

17. The computer-readable media of claim 14, wherein the patient investigation report provides visualizations of patient data comprising one or more of fall risk score, bed exit alarm status, foot rail position, head rail position, bed brake status, bed height status, head of bed angle, and patient vital sign readings.

18. A computer-implemented method of generating fall reports for a healthcare facility, the method comprising:
receiving, at a computing device, data from one or more electronic information systems and patient monitoring devices associated with the healthcare facility;
extracting falls-related data for at least one patient within the healthcare facility;
automatically generating a patient investigation report based on the falls-related data for the at least one patient;
communicating the patient investigation report to at least one computer workstation;
aggregating, at the computing device, falls-related data for all patients within the healthcare facility, including the at least one patient;
generating and displaying a user interface on the at least one computer workstation, the user interface comprising one or more parameter configuration boxes and a time configuration box;
receiving selections of report parameters through the one or more parameter configuration boxes and the time configuration box on the user interface;
filtering the aggregated falls-related data based on the selected report parameters;
automatically generating a facility investigation report based on the filtered data; and
displaying the facility investigation report on the at least one computer workstation, the facility investigation report comprising a chart area displaying one or more visualizations of the filtered data.

19. The method of claim 18, wherein the patient investigation report is automatically generated when data is received indicating that the at least one patient was involved in a fall.

20. The method of claim 18, wherein the facility investigation report is populated with anonymized patient data accessed from a data warehouse.
